(19) United States
(12) Patent Application Publication
(10) Pub. No.: US 2025/0288610 A1
(43) Pub. Date: Sep. 18, 2025

(54) Nutritional or Dietary Supplement to Enhance Regenerative Medicine Therapies

(71) Applicant: Restoration Biologics, Flagstaff, AZ (US)

(72) Inventors: Farshid Guilak, Clayton, MO (US); Suzann M. Tabbaa, Flagstaff, AZ (US)

(21) Appl. No.: 18/858,741

(22) PCT Filed: Apr. 21, 2023
(86) PCT No.: PCT/US2023/019383

§ 371 Date: Oct. 21, 2024

Related U.S. Application Data
(60) Provisional application No. 63/333,236, filed on Apr. 21, 2022.

(30) Foreign Application Priority Data
Apr. 21, 2022 (US) 63333236

(51) Int. Cl. A61K 35/14; A61K 45/06

(52) U.S. Cl. CPC A61K 35/14; A61K 45/06

(57) ABSTRACT

This invention involves a nutritional or dietary supplement composition that optimizes the beneficial factors and effects of autologous regenerative medicine therapies and non-autologous regenerative medicine therapies. The supplement is comprised of ingredients specific to the clinical application. The ingredients include various types of herbs or other botanicals, spices, amino acids, carbohydrates, dietary sources of fatty acids including various oils, ions, vitamins, antioxidants, enzymes or other dietary substance. An individual or donor is administered a tailored supplement formulation for a specific clinical indication. After the tailored supplemental formulation has been in the donor, the next step is to extract the enhanced tissue or cells or blood components, at, from the individual or donor. The supplement is administered at defined times prior to regenerative medicine therapy. The ingredients of the supplement can be provided in forms including teas, powder, tablet, liquid, or capsule for oral ingestion.

### CROSS-REFERENCE TO RELATED APPLICATIONS

This application claims the benefit of U.S. Provisional Application No. 63/333,236 filed on Apr. 21, 2022, and entitled, “Nutritional or Dietary Supplement to Enhance Regenerative Medicine Therapies”, incorporated herein by reference in its entirety.

### FIELD OF THE INVENTION

The present invention relates to a nutritional or dietary supplement that enhances autologous or non-autologous regenerative therapies.

### BACKGROUND OF THE INVENTION

Regenerative medicine therapies are used in various clinical applications to augment the healing of tissues, reduce pain, and potentially treat various pathologies and diseases. See for example, U.S. Pat. Nos. 8,603,522; 9,845,457; 10,159,705; and 10,259,772, all of which are incorporated by reference in their entirety.

Many of the clinically available treatments utilize autologous tissues and blood of bone marrow components to augment the healing of tissue repair or treat various conditions. The use of autologous tissue, blood, or bone marrow can be very inconsistent between patients. For example, autologous plasma can be processed with various systems to produce platelet rich plasma (PRP) or platelet poor plasma (PPP), or other compositions with various concentrated biological factors. The plasma extracted will vary substantially by person resulting in various compositions and concentrations of factors and ultimately varied biological potential of the product. Similarly, bone marrow aspirate (BMA) comprises various amounts stem or progenitor cells, growth factors, and other biologic components that may improve healing or mitigate pain. The cellular and molecular factors in bone marrow aspirate will differ widely between patients/individuals. Age has been shown to reduce the number of progenitor cells and function. Sex is another factor that can contribute to the number and types of cells isolated from bone marrow. Plasma based products like PRP, BMA, and other autologous tissues are frequently used in orthopedic applications to augment healing or reduce pain. For example, PRP has been used to treat osteoarthritis of various joints, tendinopathy, plantar fasciitis, and other musculoskeletal pathologies. Similarly, BMA and concentrated BMA has also been used as an adjunct to bone healing, as well as, a modality to treat osteoarthritis or other joint disorders.

Due to the variability in biological potential of the autologous plasma and bone marrow products, it has been reported that these products can vary widely in composition and function. Diet can play a significant role in enhancing the health of a patient as well as influence the effectiveness of treatment and contribute to the healing process. Diet can also influence the autologous tissues and blood components extracted for regenerative therapies. Specifically, dietary content may influence the composition of plasma used for PRP products and the cellular composition and function of bone marrow and other sources, such as adipose tissue. The effectiveness of autologous and non-autologous regenerative therapy products is limited by the patient/individual variability. Furthermore, these autologous regenerative therapies such as PRP and BMAC may contain both beneficial and deleterious factors depending on the application.

If a process could enhance the beneficial factors, reduce the detrimental factors, and thus reduce patient variability, these technologies will have better biologic potential and outcomes. Improving the patient health with diet and a tailored supplement prior, during, and after a regenerative medicine therapy may augment biological potential of the treatment by enhancing the quality of the therapy (i.e., PRP or cell therapies) and/or enhance the patient health and host environment or environment of the implant site receiving the therapy.

### SUMMARY OF THE INVENTION

This invention describes both a tailored dietary supplement to enhance a regenerative medicine therapy and a method of using the supplement to optimize the biological potential of the host and regenerative medicine therapy. The supplement composition will vary based on type of regenerative therapy and clinical application. The composition may include various dosages or combinations percent weight of fatty acids from oils, minerals, vitamins, antioxidants, botanicals, carbohydrates, or other dietary constituents or nutraceuticals. The supplement will be taken orally prior, during, and/or after a regenerative medicine therapy is administered to improve healing potential of the therapy by enhancing the therapy directly and/or the patient host environment where the regenerative medicine therapy is intended.

The method comprises optimizing the biological potential of autologous or non-autologous regenerative medicine therapies by administering a tailored dietary supplement specific for the therapy and clinical application. The dietary supplement may comprise enzymes, amino acids, minerals, botanicals, herbs, oils, fatty acids, vitamins, nutraceuticals, carbohydrates, or other dietary constituents. The dietary supplement will vary in composition and weight percentage of the dietary constituents based on clinical application and regenerative therapy used. When an autologous regenerative medicine therapy is being used the dietary supplement will be administered prior to harvest of autologous tissues. The supplement may also include food sources with anti-inflammatory compounds that can be selectively released using various enzymes. The supplement may be used in conjunction with fasting or specific diet requirement. Autologous regenerative medicine therapies could include the general use of plasma, bone marrow aspirate, iliac crest autologous bone, adipose tissue, autograft of various tissue types, or any cell-based therapy. Non-autologous regenerative medicine therapies could include cell therapy, gene therapy, therapeutic tissue engineering product, any human cell and tissue product.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows a method for enhancing a regenerative therapy from an individual or donor to treat an individual's condition or injury.

### DETAILED DESCRIPTION OF THE PREFERRED EMBODIMENTS

The following detailed description should be read with reference to the drawing. The detailed description and the drawings, which are not necessarily to scale, set forth illustrative and exemplary embodiments and are not intended to limit the scope of the disclosure. Selected features of any illustrative embodiment can be incorporated into an additional embodiment unless clearly stated to the contrary.

While the disclosure is amenable to various modifications and alternative forms, specifics thereof have been shown by way of example in the drawings and will be described in detail. As used herein in the specification and claims, including as used in the examples and unless otherwise expressly specified, all numbers may be read as if prefaced by the word “about” or “approximately,” even if the term does not expressly appear, with it being understood that this provides a reasonable expected range of values in the order of +/−10% of the stated value (or range of values). In addition, any numerical range recited herein is intended to include all sub-ranges subsumed therein. Overall, it should be understood, the intention is not to limit aspects of the disclosure to the particular illustrative embodiments described. On the contrary, the intention is to cover all modifications, equivalents, and alternatives falling within the spirit and scope of the disclosure.

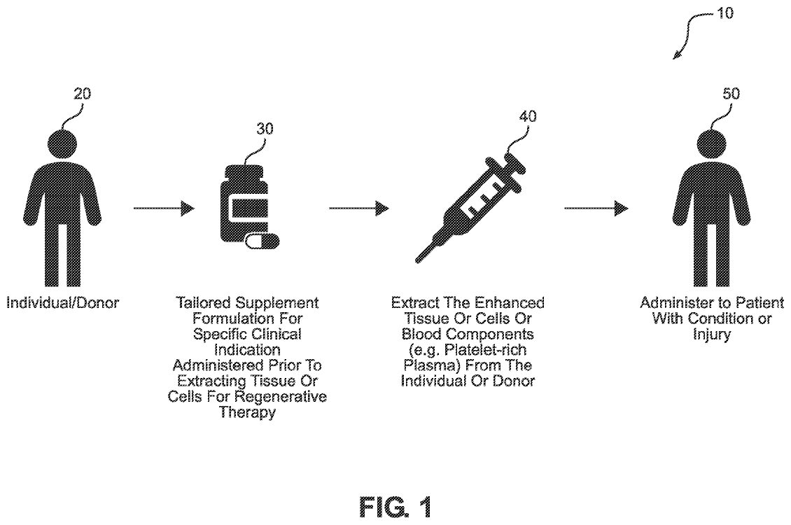

A method for enhancing a therapeutic efficacy of a regenerative therapy 10 is shown in FIG. 1. An individual or donor 20 is administered a tailored supplement formulation 30 for a specific clinical indication. Administering of the nutritional supplement formulation is preferably conducted orally, or nasally, or as an injection intravenously, intraperitoneally, intramuscularly, intra-marrow, or in some other body site. The administration of the tailored supplement formulation, which is preferably an oral nutritional supplement, will biologically enhance the donor tissue or cells or blood components of the donor used as a regenerative therapy to treat or repair a condition or injury.

Preferably the formulation 30 is specific to the condition or injury and further comprises: enzymes, amino acids, minerals, botanicals, herbs, oils, fatty acids, vitamins, nutraceuticals, carbohydrates, antioxidants, or other dietary constituents. The formulation 30 is also preferably configured to be an anti-inflammatory and further comprises fatty acids, antioxidants, or other dietary constituents. The formulation is preferably further configured to enhance an individual's blood or blood components prior to treating or repairing an inflammatory condition or injury.

After the tailored supplemental formulation has been in the donor, the next step is to extract the enhanced tissue or cells or blood components, at 40, from the individual or donor. This step 40 includes isolating a platelet-rich plasma or platelet-poor plasma that is then reintroduced or reimplanted in the individual. Other tissue could be blood, bone marrow, fat, or any other tissue or bodily fluid and preferably includes stem cells.

The enhanced tissue is then administered to a patient with a condition or injury to treat the individual's condition or injury. It should be noted that the donor 20 and the patient 50 could be the same person. Therefore, administering a nutritional supplement formulation 30 to an individual patient 50 and then isolating autologous materials at 40 could result in the materials being reintroduced or reimplanted in the donor 20. Also, administering the nutritional supplement could be conducted during and/or postoperatively to further enhance therapy.

This invention provides a method to enhance the biological potential of regenerative medicine therapies through oral administration of a dietary supplement. The supplement may be taken preoperatively and/or intraoperatively and/or postoperatively following clinical application of regenerative therapy for both autologous or non-autologous regenerative therapies. The supplement may be used in conjunction with fasting or a specific diet.

The supplement and method for administration will vary depending on the type of regenerative medicine therapy and the clinical application. The supplement may be administered orally years, months, days, or hours prior to the autologous tissue harvest. The supplement may be taken daily, twice a day, three times a day, up to 10 times a day. Depending on the type of autologous regenerative therapy, the supplement may be taken years, months, days, hours, or minutes before the autologous tissue, blood, or marrow components are harvested to optimize the biological potential of the tissues and reduce variability. The same supplement or different supplement may be taken during or post-treatment to optimize the healing potential of the patient and enhance the host environment where the therapy is intended.

If autologous blood components or marrow components are used as a regenerative medicine therapy for treating an inflammatory condition such as osteoarthritis, tendinopathy, degenerative disc disease, etc., the supplement may include the dietary constituents with anti-inflammatory properties including but not limited to specific fatty acids (e.g., omega-3 or omega-9 polyunsaturated fatty acids or odd chain fatty acids), oils from plants, curcumin, zinc, green tea, frankincense, antioxidants, dietary sources of polyphenols, glucocorticoids, and vitamins. The supplement may require fasting for 12-24 hours prior to use. If autologous blood components or marrow components are used for wound healing or bone healing the following constituents preferably comprise the supplement: minerals such as amino acids, iron, manganese, zinc, copper, and vitamins such as vitamin A, K, C, B6. When a non-autologous regenerative medicine therapy is being applied to a patient, a dietary supplement is preferably applied before, during, or after administering treatment.

The constituents of the supplement will vary based on the clinical application and pathology to enhance or support the effect of the regenerative therapy by enhancing the patient health and host environment where the therapy is applied. When a cell therapy is applied to reduce inflammation, a supplement with various constituents and weight percentage may be applied to stimulate the cells and promote healing. The supplement preferably includes dietary constituents with anti-inflammatory properties including but not limited to specific fatty acids (e.g., omega-3 or omega-9 polyunsaturated fatty acids), oils from plants, curcumin, zinc, green tea, frankincense, antioxidants, dietary sources of polyphenols, glucocorticoids, enzymes, and vitamins.

In one preferred embodiment, the regenerative medicine therapy includes autologous tissues, cell, or molecular factors harvested from a patient. Examples include iliac crest autologous bone graft, fat graft, bone marrow aspirate, blood components such as plasma, adult cells from various tissue types (e.g., cartilage or tendon) or stem cells from a variety of potential donor sites (bone marrow, adipose tissue, blood). If autologous tissues or component are harvested for a regenerative medicine therapy, a supplement with dietary constituents specific for the clinical application will be applied prior to tissue harvest to optimize the composition of the autologous tissue.

The supplement comprises various constituents including minerals, enzymes, vitamins, oils or fatty acids from various sources, botanicals, herbs, and other dietary components or nutraceuticals depending on the application. The constituents are preferably combined with enzymes to selectively release beneficial components. The supplement is preferably in various forms such as liquids, tablets, powder, capsules to be taken orally.

The supplement may be taken daily years, months, days, or hours prior to autologous tissue harvest. The supplement may be taken once a day, twice a day, or up to 10 times a day. The supplement may be taken hours or minutes before extracting autologous tissues, cells, blood, or marrow components depending on the type of tissue and regenerative medicine therapy. The supplement may be taken during and/or postoperatively to further enhance therapy and/or the host environment where the therapy is intended.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

1. A nutritional supplement comprising a formulation configured to biologically enhance donor biologic material used as a regenerative therapy to treat or repair a condition or injury.

2. A nutritional supplement of claim 1, wherein the formulation is specific to the condition or injury and further comprises: enzymes, amino acids, minerals, botanicals, herbs, oils, fatty acids, vitamins, nutraceuticals, carbohydrates, antioxidants, or other dietary constituents.

3. A nutritional supplement of claim 2, wherein the formulation is configured to be an anti-inflammatory and further comprises fatty acids, antioxidants, or other dietary constituents and is further configured to enhance an individual's blood or blood components prior to treating an inflammatory condition or injury.

4. A nutritional supplement of claim 1, wherein the biologic material is tissue, cell, organ, or bodily fluid derived.

5. A method of enhancing regenerative therapy comprising:
providing an oral nutritional supplement to an individual prior to a regenerative therapy derived from that individual, wherein the nutritional supplement includes a formulation configured to enhance a therapeutic efficacy of therapy.

6. The method of claim 5, wherein the formulation contains one or more of: enzymes, amino acids, minerals, botanicals, herbs, oils, fatty acids, vitamins, nutraceuticals, carbohydrates, or other dietary constituents.

7. The method of claim 5, further comprising:
administering the nutritional supplement formulation orally, or nasally, or as an injection intravenously, intraperitoneally, intramuscularly, intra-marrow, or in some other body site.

8. The method of claim 5, further comprising administering the nutritional supplement during and/or postoperatively to further enhance therapy.

9. The method of claim 5, further comprising:
administering a nutritional supplement to an individual; and
isolating biological materials that are then reintroduced or reimplanted in the individual.

10. The method of claim 6, further comprising:
administering a nutritional supplement to an individual,
isolating blood or blood components, and
reintroducing or reimplanting the blood or blood components into the individual.

11. The method of claim 10, wherein isolating the blood or blood components includes isolating blood or blood components that is then reintroduced or reimplanted in the individual.

12. The method of claim 6, further comprising administering a nutritional supplement to an individual before isolation of a living material, such as blood, bone marrow, fat, or any other tissue or bodily fluid, that is then reintroduced or reimplanted in the individual to treat a condition or injury.

13. The method of claim 6, further comprising administering a nutritional supplement to an individual before isolation of living cells, including stem cells from blood, bone marrow, fat, or any other tissue or bodily fluid, that are then reintroduced or reimplanted in the individual.

14. The method of claim 6, where administering the nutritional supplement to the individual continues for a duration of time ranging from at the time of regeneration therapy to 1 year prior to regenerative therapy.

15. The method of claim 5, wherein the nutritional supplement administered to a donor individual separate from the individual receiving the therapy.
